(19) United States
(12) Patent Application Publication
(10) Pub. No.: US 2013/0085111 A1
(43) Pub. Date: Apr. 4, 2013

(54) PRODUCTION OF HUMAN C1 INHIBITOR IN HUMAN CELLS

(71) Applicants: Victor Gurewich, Cambridge, MA (US); Alexis Wallace, Cambridge, MA (US)

(72) Inventors: Victor Gurewich, Cambridge, MA (US); Alexis Wallace, Cambridge, MA (US)

(73) Assignee: Thrombolytic Science, LLC, Cambridge, MA (US)

(21) Appl. No.: 13/634,776

(22) PCT Filed: Mar. 18, 2011
(86) PCT No.: PCT/US11/29011

§ 371 Date: Dec. 3, 2012

Related U.S. Application Data
(60) Provisional application No. 61/315,282, filed on Mar. 18, 2010.

(51) Int. Cl. C12P 21/00

(52) U.S. Cl. CPC C12P 21/005
USPC 514/20.9; 435/366; 530/395; 435/455; 435/69.2

(57) ABSTRACT

Vector constructs comprising the coding sequence for human C1 inhibitor are described. Expression of glycosylated recombinant human C1 inhibitor is achieved human cells in high yields.

### FIELD OF THE INVENTION

The invention relates to the production of human C1 inhibitor or portions thereof in human cells. Expression of recombinant human C1 inhibitor is achieved in high yields.

### BACKGROUND OF THE INVENTION

Human C1 inhibitor, also known as C1 esterase inhibitor, is a well-known and identified substance. C1 inhibitor belongs to the superfamily of serine proteinase inhibitors and is the only inhibitor of C1r and C1s of the complement system and is the major inhibitor of factor XIIa and kallikrein. In addition C1 inhibitor inhibits also other serine proteases of the coagulation and fibrinolytic systems like factor XI, tissue type plasminogen activator and plasmin (Schapira M. et al. 1985, Complement 2:111/Davis A. E. 1988, Ann. Rev. Immunol. 6:595).

C1 inhibitor is encoded by a single gene on chromosome 11 and consists of 8 exons and 7 introns. The entire genomic sequence is known and codes for a protein of 500 amino acids, including a 22 amino acid signal sequence (Carter P. et al. 1988, Euro. J. Biochem. 173; 163). Plasma C1 inhibitor is a glycoprotein of approximately 105 kDa and is heavily glycosylated, up to 50% of its molecular mass consists of carbohydrate.

C1 inhibitor obtained from human blood, either highly or partially purified, is used and approved in some European countries for the treatment of hereditary angioedema. However, product isolated from plasma poses substantial risk of contamination. The plasma preparations of C1 inhibitor used at present are vapor-treated or pasteurized products. The heat treatment is a precaution to eliminate blood born infectious agents. Although taking the precautions for virus removal/inactivation there is still a risk for transmission of viruses such as HIV and hepatitis (De Filippi F. et al. 1998, Transfusion 38: 307). In addition to the safety problem the lack of availability of purified plasma C1 inhibitor as well as the high costs involved are disadvantages.

The production of functional C1 inhibitor in COS or CHO cells via recombinant DNA technology has been reported (see e.g. Eldering E. et al. 1988, J. Biol. Chem. 263: 11776). However, the reported yield in the ug/ml range is too low for therapeutic application.

The production of human C1 inhibitor in a baculovirus expression vector system has been described. Wolff et al., Protein Express. & Purif 22: 414-421 (2001). However, the glycosylation of the protein is quite different from normal human C1 inhibitor.

The production of an active human C1 inhibitor in bacteria has been described, even though the truncated fragment was not glycosylated. Lamark et al., Protein Express. & Purif. 22: 349-358 (2001). However, a major obstacle to large-scale production was the fact that much of the protein was produced in an insoluble form.

Clearly, safe and effective human C1 Inhibitor preparations are needed for human administration.

### SUMMARY OF THE INVENTION

The present invention contemplates, in one embodiment, producing a recombinant human C1 inhibitor or portion thereof (e.g. the Serpin domain) in human cells. In one embodiment, the C1 inhibitor or portion thereof is part of a fusion protein. It is not intended that the present invention be limited by the human cell type. However, in a preferred embodiment, the human cells are Human Embryonic Kidney 293 cells, also often referred to as HEK 293, 293 cells, or less precisely as HEK cells. These cells (whether pre- or post-transfection) can be grown as monolayers or in suspension cultures.

The present invention contemplates vectors, host cells, transfected host cells, expressing host cells, expressed protein that is glycosylated, and purified expressed protein. For example, the present invention contemplates a human host cell comprising an expression vector (e.g. transformed cells), said vector encoding human C1 inhibitor or a portion thereof. In one embodiment, said host cell is capable of expressing said human C1 inhibitor or portion thereof as a soluble protein at a level greater than or equal to 0.75% of the total cellular protein. In one embodiment, said host cell is capable of expressing said human C1 inhibitor or portion thereof as a soluble protein at a level greater than or equal to 5% of the total cellular protein. In one embodiment, said host cell is capable of expressing said human C1 inhibitor or portion thereof as a soluble protein at a level greater than or equal to 15% of the total cellular protein. In one embodiment, said vector encodes a portion consisting of the Serpin domain of human C1 inhibitor. In one embodiment, said vector encodes a fusion protein comprising at least a portion of human C1 inhibitor, said portion comprising a portion of the sequence of SEQ ID NO:1. In one embodiment, said fusion protein comprises a poly-histidine tract. In one embodiment, the host cells are HEK 293 cells.

The present invention also contemplates a soluble fusion protein comprising at least a portion of glycosylated human C1 inhibitor, said portion comprising a portion of the sequence of SEQ ID NO:1. In one embodiment, said portion consists of the Serpin domain of human C1 inhibitor. In one embodiment, said fusion protein comprises a poly-histidine tract. In one embodiment, said fusion protein is substantially endotoxin-free.

The present invention also contemplates a method, comprising: a) providing human cells and an expression vector, said vector encoding human C1 inhibitor or a portion thereof; b) introducing said expression vector into said human cells under conditions such that said human cells glycosylate and express human C1 inhibitor protein or a portion thereof (i.e. an N-glycosylated C1 inhibitor protein). In one embodiment, the method further comprises c) culturing said cells under conditions such that said human C1 inhibitor protein or portion thereof is expressed at a level of at least 20 mg/L (and more preferably, at least 30 mg/L) in the supernatant. In one embodiment, said human C1 inhibitor protein or portion thereof is expressed at a level between 30 mg/L and 50 mg/L. In one embodiment, the method further comprises d) purifying said human C1 inhibitor protein or portion thereof so as to prepare purified product. In one embodiment, said purified product has an apparent molecular weight on SDS-PAGE of greater than 100 kDa. In one embodiment, said purifying comprises column chromatography (e.g. with affinity resins and/or specific antibody). In one embodiment, said human cells are HEK 293 cells. In one embodiment, the method further comprises e) administering said purified product to a human subject. In one embodiment, said human subject is a patient. The present invention also contemplates in one embodiment, the purified glycosylated recombinant human C1 inhibitor made by the above-described method.

### DEFINITIONS

In one embodiment, the present invention contemplates fusion proteins and methods of making fusion proteins. As used herein, the term “fusion protein” refers to a chimeric protein containing the protein of interest (i.e., C1 Inhibitor or fragments thereof) joined to an exogenous protein fragment (the fusion partner which consists of another protein or protein fragment). The fusion partner may enhance solubility of the C1 inhibitor protein or protein fragment as expressed in a (preferably human) host cell, and may also provide an affinity tag to allow purification of the recombinant fusion protein from the host cell or culture supernatant, or both. If desired, the fusion protein may be removed from the protein of interest prior to administration by a variety of enzymatic or chemical means known to the art.

The present invention contemplates recombinant human C1 inhibitor. The term “recombinant” as used herein refers to a protein molecule expressed from a recombinant DNA molecule (e.g. an expression vector comprising an inserted sequence coding for the protein). In contrast, the term “native protein” is used herein to indicate a protein isolated from a naturally occurring (i.e., a non-recombinant) source. Molecular biological techniques may be used to produce a recombinant form of a protein with identical properties as compared to the native form of the protein. Expression vectors can be introduced into cells by transfection. The term “transfection” as used herein refers to the introduction of foreign DNA into eukaryotic cells. Transfection may be accomplished by a variety of means known to the art including calcium phosphate-DNA co-precipitation, DEAE-dextran-mediated transfection, polybrene-mediated transfection, electroporation, microinjection, liposome fusion, lipofection, biolistics and the like. Such “transformed” cells include stably transformed cells in which the inserted DNA is capable of replication either as an autonomously replicating plasmid or as part of the host chromosome. They also include cells which transiently express the inserted DNA or RNA for limited periods of time.

As used herein in reference to an amino acid sequence or a protein, the term “portion” (as in “human C1 inhibitor or a portion thereof”) refers to fragments of that protein. The fragments may range in size from four amino acid residues to the entire amino acid sequence minus one amino acid. Portions of C1 inhibitor include (but are not limited to) the full-length protein minus the signal peptide, the Serpin domain, and the like.

As used herein, the temi “poly-histidine tract” when used in reference to a fusion protein refers to the presence of two to ten histidine residues at either the amino- or carboxy-terminus of a protein of interest, i.e. C1 inhibitor or portion thereof (e.g. the Serpin domain). A poly-histidine tract of six to ten residues is preferred. The poly-histidine tract is also defined functionally as being a number of consecutive histidine residues added to the protein of interest which allows the affinity purification of the resulting fusion protein on a nickel-chelate column.

As used herein, the terms “purifying,” “purified” or “to purify” means the process or result of any process that removes some of a contaminant from the component of interest, such as a protein or nucleic acid. The percent of a purified component or “purified product” is increased in the sample. The term is not limited to the situation where all contaminants are removed completely. Purification can be done by centrifugation (e.g. to remove contaminating cells) or by more extensive methods such as ion exchange chromatography (e.g. anion exchange chromatography with an ion exchange resin such as SP Sepharose), affinity chromatography or size exclusion chromatography.

The tenn “subject” includes humans and non-human animals. In the case of humans, the term includes more than patients.

### DESCRIPTION OF THE FIGURES

FIG. 1 shows the human C1 Inhibitor amino acid sequence (including the peptide signal) (SEQ ID NO:1).

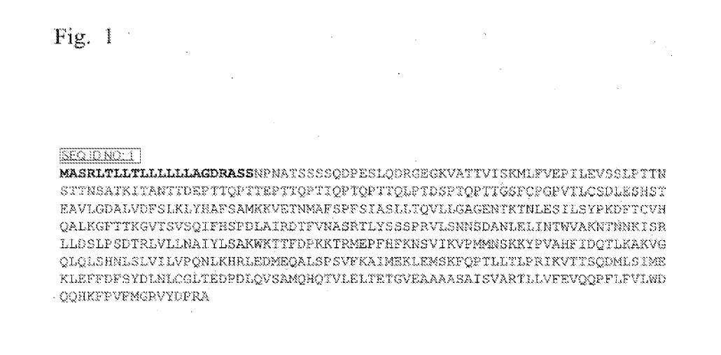

FIG. 2 shows the nucleotide sequence of the nucleic acid encoding full length human C1 Inhibitor (SEQ ID NO:2).

FIG. 3 is a schematic showing one embodiment of an exemplary bacterial plasmid for inserting the coding sequence.

FIG. 4 is a schematic showing one embodiment of an exemplary vector and sequencing primers (SEQ ID NOS: 3, 4 and 5).

FIG. 5 is a schematic showing one embodiment of the exemplary expression vector with the coding sequences for C1 Inhibitor inserted.

FIG. 6 is a photograph of a Cocmassie blue stained SDS-PAGE gel where the proteins in the cell supernatant are compared in intensity with a standard.

FIG. 7 is a photograph of a Western blot (after SDS-PAGE) of harvested supernatants from cultures of transfected human cells expressing recombinant glycosylated human C1 inhibitor.

### DETAILED DESCRIPTION

Human C1 inhibitor is a highly glycosylated serine protease inhibitor of the serpin family. The protein contains two disulfide bonds. In one embodiment, the present invention contemplates expressing and producing full length human C1 inhibitor in human cells. In another embodiment, the present invention contemplates expressing and producing a portion or fragment thereof (e.g. an N-terminally truncated form of recombinant C1 inhibitor). Ideally, recombinant proteins are expressed as soluble proteins at high levels (i.e., greater than or equal to about 0.75% of total cellular protein, and more preferably, greater than 5% or even 15% of total cellular protein) in host cells. Said another way, in a preferred embodiment, human cells comprising an expression vector are cultured under conditions such that glycosylated human C1 inhibitor is expressed at a level greater than or equal to 30 mg/L. This facilitates the production and isolation of sufficient quantities in a highly purified form (i.e., substantially free of endotoxin or other pyrogen contamination).

In one embodiment, the present invention contemplates expressing and producing a C1 Inhibitor or C1 Inhibitor fragment comprising a poly-histidine tract (also called a histidine tag). In a particularly preferred embodiment, a fusion protein comprising the histidine tagged Serpin domain. The production of C1 inhibitor or C1 inhibitor fragment fusion proteins containing a histidine tract is not limited to the use of a particular expression vector and host strain. Several commercially available expression vectors and host strains can be used to express the C fragment protein sequences as a fusion protein containing a histidine tract. For example, Qiagen has a pQE xpression vector for mammalian cells that is commercially available.

A variety of routes of administration may be used. However, it is preferred that administration of the recombinant protein (or fragment thereof) be done intravenously.

### Experimental

### Example 1

### Construction of Inserts and Vectors

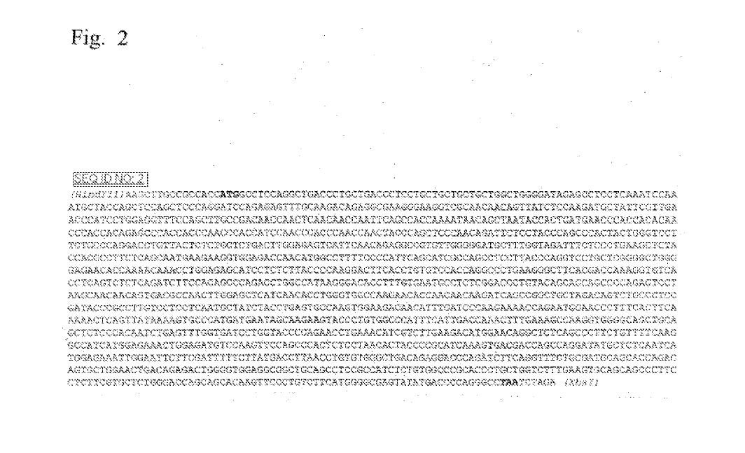

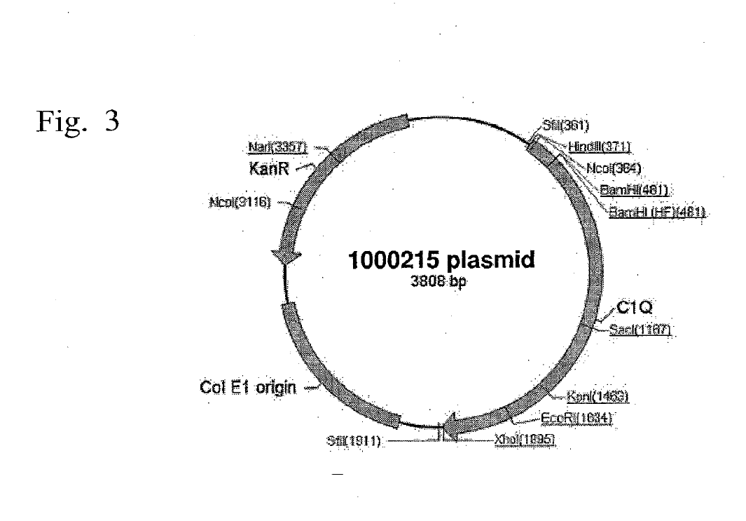

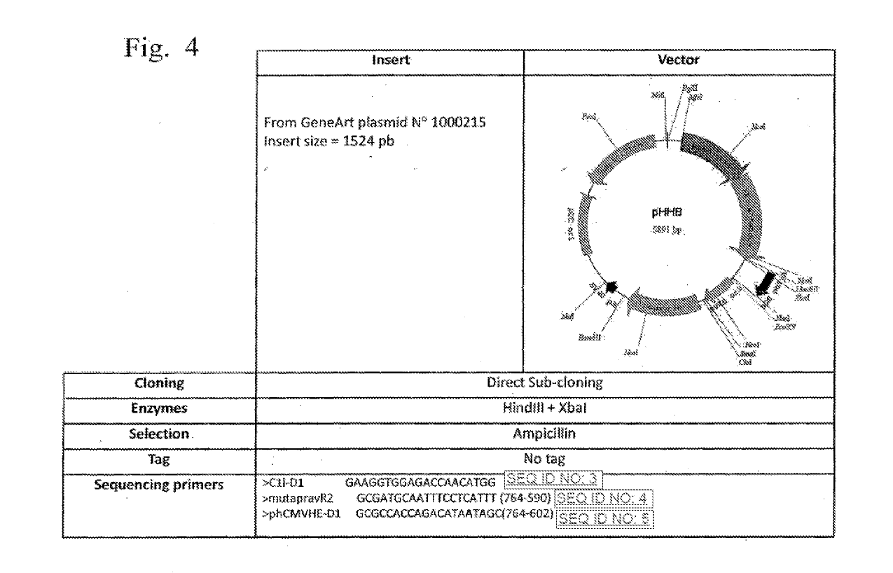

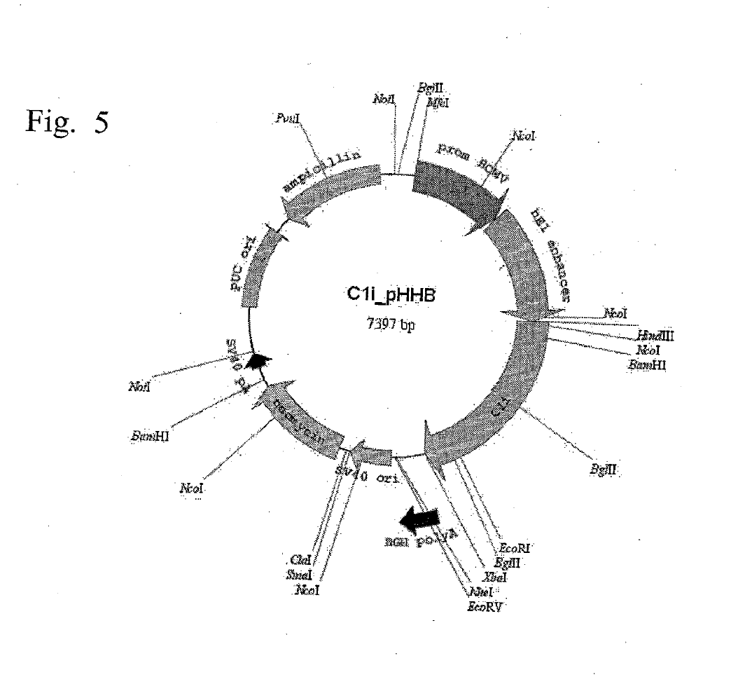

The gene for human C1 inhibitor (see coding sequence in FIG. 2) was assembled from synthetic oligonucleotides and PCR products. The fragment was cloned into pMK-RQ (kanR) using SfilI restriction sites (FIG. 3). The plasmid DNA was purified from transformed bacteria and was verified by sequencing to assess the absence of mutations. A 1524 by insert from the plasmid was inserted into a pHHB vector (FIG. 4) for subcloning using Ampicillin selection (the vector with the insert is shown in FIG. 5). Sequencing with sequencing primers was done with the plasmid DNA from 4 clones in order to identify one construct with the expected sequence.

### Example 2

### Transfection of Human Cells and Expression of Human C1 Inhibitor Proteins

HEK 293 “Freestyle” cells (Invitrogen Corp.) were amplified until a concentration of 0.7 x 10⁶ cells/ml. Transfection of these cells was performed uing 293fectin and 50 ug C1-pHHB/1 plasmid in 50 ml volumes. The cells were cultured and 1 ml of culture raw supernatant was harvested and centrifuged (200g for 15 minutes). After centrifugation, the purified supernatant was taken and stored at 20C with 1mM Leupeptin, 1 mM Pepstatin, 1 mM PMSF and 10% glycerol to ensure stability.

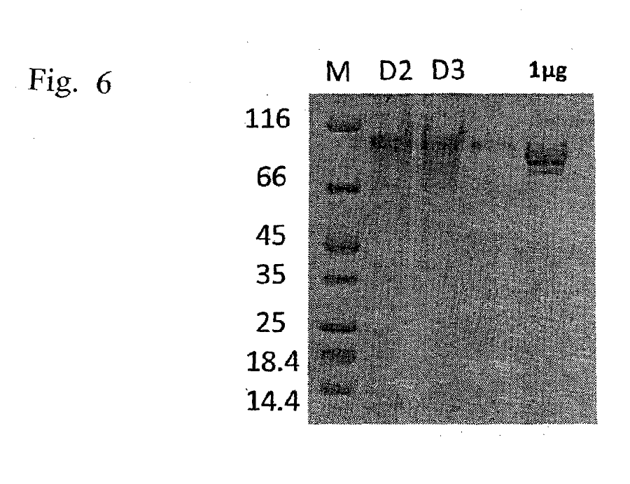

For analysis, harvested supernatants [i.e. after centrifugation (D2) or after centrifugation and the addition of stabilizers (D3)] were loaded on an SDS-PAGE gel (30 ul well) and the intensity of the protein bands were compared with a standard of known protein concentration (i.e. 1 ug) stained with Coomassie blue. FIG. 6 shows the gel results, permitting an estimation that an expression of approximately 30 mg/L or greater of C1 Inhibitor was achieved.

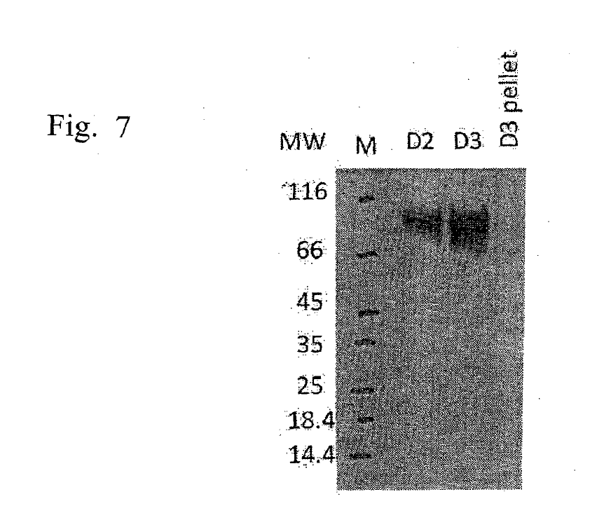

After SD S-PAGE, the proteins were transferred to a membrane by Western blotting. The membrane was reacted with a commercially available antibody (Anti-human Serpin G1/C1 inhibitor antibody from R&D Systems) at a 1/1000 dilution. The results (FIG. 7) show the antibody reacting with both supernatant preparations (D2 and D3). Based on the molecular weight markers, the apparent molecular weight is greater than 100kDa (approx. 104-106kDa) indicating glycosylation.

N-glycosylation of C1 Inhibitor was investigated using an N-deflycosylation Kit (Glycoprofile II, Sigma). The results (not shown) indicate that the recombinant human C1 Inhibitor is N-glycosylated.

### Example 3

### Purification of Recombinant Glycosylated Human C1 Inhibitor Protein

Anion exchange chromatography was chosen for purification of cell culture supernatants containing recombinant glycosylated human C1 inhibitor. 30 ml of cell supernatant was dialyzed against 20 mM Sodium Phosphate pH 7.0. 1 ml of SP Sepharose high performance was chosen as the ion-exchange resin. The equilibration buffer was 20 mM Sodium Phosphate pH 7.0. The Elution buffer was 20 mM Sodium Phosphate pH 7.0, 500 mM NaCl. The dialyzed supernatant was added to the resin and eluted with a gradient from 0 to 100% of the elution buffer on 30 CV. Analysis on SDS -PAGE (not shown) showed good capture of C1 inhibitor.

The purification was scaled up to 500 ml supernatant and 4 ml resin. The dialyzed supernatant was added to the resin and eluted by steps (10, 15, 28, 36, 50 and 100% elution buffer). Again, analysis by SDS-PAGE (not shown) showed good capture of the C1 inhibitor protein.

1. A human host cell comprising an expression vector, said vector encoding human C1 inhibitor or a portion thereof.

2. The human host cell of claim 1, wherein said host cell is capable of expressing said human C1 inhibitor or portion thereof as a soluble protein at a level greater than or equal to 0.75% of the total cellular protein.

3. The human host cell of claim 1, wherein said host cell is capable of expressing said human C1 inhibitor or portion thereof as a soluble protein at a level greater than or equal to 5% of the total cellular protein.

4. The human host cell of claim 1, wherein said host cell is capable of expressing said human C1 inhibitor or portion thereof as a soluble protein at a level greater than or equal to 15% of the total cellular protein.

5. The human host cell of claim 1, wherein said vector encodes a portion consisting of the Serpin domain of human C1 inhibitor.

6. The human host cell of claim 1, wherein said vector encodes a fusion protein comprising at least a portion of human C1 inhibitor, said portion comprising a portion of the sequence of SEQ ID NO:1.

7. The human host cell of claim 6, wherein said fusion protein comprises a poly-histidine tract.

8. A soluble fusion protein comprising at least a portion of glycosylated human C1 inhibitor, said portion comprising a portion of the sequence of SEQ ID NO:1.

9. The fusion protein of claim 8, wherein said portion consists of the Serpin domain of human C1 inhibitor.

10. The fusion protein of claim 8, wherein said fusion protein comprises a poly-histidine tract.

11. The fusion protein of claim 8, wherein said fusion protein is substantially endotoxin-free.

12. A method, comprising:
a) providing human cells and an expression vector, said vector encoding human C1 inhibitor or a portion thereof;
b) introducing said expression vector into said human cells under conditions such that said human cells express and glycosylate human Cl inhibitor protein or a portion thereof.

13. The method of claim 12, further comprising c) culturing said cells under conditions such that said human C1 inhibitor protein or portion thereof is expressed at a level of at least 30 mg/L.

14. The method of claim 13, wherein said human C1 inhibitor protein or portion thereof is expressed at a level between 30 mg/L and 50 mg/L.

15. The method of claim 14, further comprising d) purifying said human C1 inhibitor protein or portion thereof so as to prepare purified product.

16. The method of claim 15, wherein said purified product has an apparent molecular weight on SDS-PAGE of greater than 100 kDa.

17. The method of claim 15, wherein said purifying comprises anion exchange chromatography.

18. The method of claim 12, wherein said human cells are Human Embryonic Kidney 293 cells.

19. The method of claim 15, further comprising e) administering said purified product to a human subject.

20. The method of claim 19, wherein said human subject is a patient.

21. The purified human C1 inhibitor made by the process of claim 15.


SEQUENCE LISTING (5 sequences)

SEQ ID NO: 1 - protein, 500 aa, Homo sapiens
MASRLTLLTLLLLLLAGDRASSNPNATSSSSQDPESLQDRGEGKVATTVISKMLFVEPILEVSSLPTTNSTTNSATKITANTTDEPTTQPTTEPTTQPTIQPTQPTTQLPTDSPTQPTTGSFCPGPVTLCSDLESHSTEAVLGDALVDFSLKLYHAFSAMKKVETNMAFSPFSIASLLTQVLLGAGENTKTNLESILSYPKDFTCVHQALKGFTTKGVTSVSQIFHSPDLAIRDTFVNASRTLYSSSPRVLSNNSDANLELINTWVAKNTNNKISRLLDSLPSDTRLVLLNAIYLSAKWKTTFDPKKTRMEPFHFKNSVIKVPMMNSKKYPVAHFIDQTLKAKVGQLQLSHNLSLVILVPQNLKHRLEDMEQALSPSVFKAIMEKLEMSKFQPTLLTLPRIKVTTSQDMLSIMEKLEFFDFSYDLNLCGLTEDPDLQVSAMQHQTVLELTETGVEAAAASAISVARTLLVFEVQQPFLFVLWDQQHKFPVFMGRVYDPRA

SEQ ID NO: 2 - DNA, 1524 bp, Homo sapiens
aagcttgccgccaccatggcctccaggctgaccctgctgaccctcctgctgctgctgctggctggggatagagcctcctcaaatccaaatgctaccagctccagctcccaggatccagagagtttgcaagacagaggcgaagggaaggtcgcaacaacagttatctccaagatgctattcgttgaacccatcctggaggtttccagcttgccgacaaccaactcaacaaccaattcagccaccaaaataacagctaataccactgatgaacccaccacacaacccaccacagagcccaccacccaacccactatccaacccacccaaccaactacccagctcccaacagattctcctacccagcccactactgggtccttctgcccaggacctgttactctctgctctgacttggagagtcattcaacagaggccgtgttgggggatgctttggtagatttctccctgaagctctaccacgccttctcagcaatgaagaaggtggagaccaacatggccttttccccattcagcatcgccagcctccttacccaggtcctgctcggggctggggagaacaccaaaacaaacctggagagcatcctctcttaccccaaggacttcacctgtgtccaccaggccctgaagggcttcacgaccaaaggtgtcacctcagtctctcagatcttccacagcccagacctggccataagggacacctttgtgaatgcctctcggaccctgtacagcagcagccccagagtcctaagcaacaacagtgacgccaacttggagctcatcaacacctgggtggccaagaacaccaacaacaagatcagccggctgctagacagtctgccctccgatacccgccttgtgctcctcaatgctatctacctgagtgccaagtggaagacaacatttgatcccaagaaaaccagaatggaaccctttcacttcaaaaactcagttataaaagtgcccatgatgaatagcaagaagtaccctgtggcccatttcattgaccaaactttgaaagccaaggtggggcagctgcagctctcccacaatctgagtttggtgatcctggtaccccagaacctgaaacatcgtcttgaagacatggaacaggctctcagcccttctgttttcaaggccatcatggagaaactggagatgtccaagttccagcccactctcctaacactaccccgcatcaaagtgacgaccagccaggatatgctctcaatcatggagaaattggaattcttcgatttttcttatgaccttaacctgtgtgggctgacagaggacccagatcttcaggtttctgcgatgcagcaccagacagtgctggaactgacagagactggggtggaggcggctgcagcgtccgccatctctgtggcccgcaccctgctggtctttgaagtgcagcagcccttcctcttcgtgctctgggaccagcagcacaagttccctgtcttcatggggcgagtatatgaccccagggcctaatctaga

SEQ ID NO: 3 - DNA, 20 bp, Artificial sequence
  Synthetic
gaaggtggagaccaacatgg

SEQ ID NO: 4 - DNA, 20 bp, Artificial sequence
  Synthetic
gcgatgcaatttcctcattt

SEQ ID NO: 5 - DNA, 20 bp, Artificial sequence
  Synthetic
gcgccaccagacataatagc